(19) United States
(12) Patent Application Publication
(10) Pub. No.: US 2021/0371511 A1
(43) Pub. Date: Dec. 2, 2021

(54) USE OF CANAKINUMAB

(71) Applicant: NOVARTIS AG, Basel (CH)

(72) Inventors: Linda MINDEHOLM, Bettlach (FR); Jens PRAESTGAARD, Carlisle, PA (US); Mattias SCHIEKER, Basel (CH)

(21) Appl. No.: 17/053,602

(22) PCT Filed: Aug. 24, 2018
(86) PCT No.: PCT/IB2018/056455

§ 371 Date: Nov. 6, 2020

Related U.S. Application Data
(60) Provisional application No. 62/669,071, filed on May 9, 2018.

(51) Int. Cl. C07K 16/24; A61P 19/02

(52) U.S. Cl. CPC C07K 16/245; C07K 2317/21; C07K 2317/76; A61P 19/02

(57) ABSTRACT

Use of an IL-1β inhibitor such as canakinumab for the treatment and/or prevention of osteoarthritis and complications related thereto.

### TECHNICAL FIELD

The present disclosure relates to novel uses and methods for reducing the risk of osteoarthritis and complications related thereto, generally comprising administering a therapeutic amount of an IL-1β inhibitor, such as a binding antibody or a functional fragment exemplified by canakinumab.

### BACKGROUND OF THE DISCLOSURE

Osteoarthritis (“OA”) is one of the most common chronic health conditions and a leading cause of pain and disability among adults. It is a degenerative, chronic, progressive, painful joint disease. Currently, there is no treatment targeting the prevention of degeneration related to OA (“DMOAD”). Hip/knee OA affects 240 million people globally. Worldwide estimates of OA are that 9.6% of men and 18.0% of women aged over 60 years have OA or symptoms associated therewith. In addition, the prevalence of OA will steadily increase and is expected to be the single greatest cause of disability in the general population by 2030. Furthermore, there are serious complications that arise with OA. The degenerative nature of the disease leads to many complications. For example, in the US in 2010 there were 7.2 million people requiring total hip/knee replacement surgery. Thus, there is an unmet medical need for treatment to reduce progression of OA and adverse events associated thereof.

### SUMMARY OF THE DISCLOSURE

Inflammation contributes to all phases of the atherothrombotic process and patients with elevated inflammatory biomarkers such as hsCRP and IL-6 have increased vascular risk despite use of aggressive secondary prevention strategies. The present disclosure relates, in part, to the finding that direct inhibition of inflammation by administration of an IL-1 beta antagonist, such as canakinumab, reduces the risk of or prevents disease progression of OA, reduces adverse events (“AE”) associated with OA, and reduces the overall need for total joint replacements (“TJR”).

Accordingly, the present invention is directed to a method of preventing or reducing the AEs associated with OA.

The present invention is also directed to a method of reducing the risk of the need of TJR in patients with OA.

Accordingly, the present invention is also directed to canakinumab for use in reducing the risk of progression of OA, the risk of needing TJR in patients with OA, and/or the risk of an AE associated with OA.

The present invention is further directed to the canakinumab for the manufacture of a medicament for reducing the risk OA, the risk of needing TJR in patients with OA, and/or the risk of an AE associated with OA.

The present invention is also directed to the use of canakinumab for the manufacture of a medicament for reducing the risk of OA, the risk of needing TJR in patients with OA, and/or the risk of an AE associated with OA.

The present disclosure is exemplified by the numbered embodiments set forth below:

1. A method for reducing risk of progression of osteoarthritis (“OA”) and/or reducing adverse events associated with OA in a patient, comprising administering an IL-1beta antagonist, wherein said patient has a high sensitivity C-reactive protein (hsCRP) level of ≥2 mg/L or greater than or equal to 3 mg/l assessed before first administration of an IL-1beta antagonist, and wherein said patient has a reduced hsCRP level of <2.3 mg/L assessed at a predetermined time point after the first administration of said IL-1beta antagonist.

2. A method for reducing risk of progression of OA and/or reducing adverse events associated with OA in a patient, comprising administering an IL-1beta antagonist, wherein said patient has a high sensitivity C-reactive protein (hsCRP) level of ≥2 mg/L assessed before first administration of an IL-1beta antagonist, and wherein said patient will continue to receive IL-1beta antagonists, provided said patient has a reduced hsCRP level of <2.3 mg/L assessed at a predetermined time point after first administration of said IL-1beta antagonist.

3. A method for reducing risk of progression of OA and/or reducing adverse events associated with OA in a patient, comprising administering canakinumab, wherein said patient has a high sensitivity C-reactive protein (hsCRP) level of ≥2 mg/L assessed before first administration of canakinumab, and wherein said patient has a reduced hsCRP level of <2.3 mg/L assessed at about 3 months or more after the first administration of canakinumab.

4. A method for reducing risk of progression of OA and/or reducing adverse events in a patient, comprising administering canakinumab, wherein said patient has a high sensitivity C-reactive protein (hsCRP) level of ≥2 mg/L assessed before first administration of canakinumab, and wherein said patient will continue to receive canakinumab, provided said patient has a reduced hsCRP level of <2.3 mg/L assessed at about 3 months or more after the first administration of canakinumab.

5. The method of any of the preceding embodiments, wherein said progression of OA include joint replacement.

6. The method of any of the preceding embodiments, wherein said patient has documented and/or symptomatic OA.

7. The method of any of the preceding embodiments, comprising administering 150 mg to 300 mg canakinumab.

8. The method according to any of the preceding embodiments, comprising administering 150 mg canakinumab.

9. The method according to any of the preceding embodiments, comprising administering 150 mg canakinumab approximately every 3 months.

10. The method according to any of the preceding embodiments, wherein the reduced level of hsCRP assessed approximately 3 months after first administration of canakinumab or after a predetermined timepoint after first administration of an IL-1beta antagonist is <1.5 mg/L.

11. The method according to any of the preceding embodiments, wherein the reduced level of hsCRP assessed approximately 3 months after first administration of canakinumab or after a predetermined timepoint after first administration of an IL-1beta antagonist is <1.0 mg/L

12. The method according to any of the preceding embodiments, wherein the reduced level of hsCRP assessed approximately 3 months after first administration of canakinumab or after a predetermined timepoint after first administration of an IL-1beta antagonist is <2.2, <2.1, <2.0, <1.9, <1.8, <1.7, <1.6, <1.5, <1.4, <1.3, <1.2, <1.1, <1.0, <0.9, <0.8, <0.7, <0.6, or <0.5 mg/L.

13. The method according to any of the preceding embodiments, wherein the documented OA has been assessed using X-ray and/or MRI.

14. The method according to any of the preceding embodiments, wherein the symptomatic evidence of OA is pain and/or impaired function.

15. The method according to any of the preceding embodiments, wherein the patient is not eligible for surgery.

16 The method according to any of the preceding embodiments, wherein the patient is not responsive to NSAIDs.

17. The method according to any of the preceding embodiments, wherein the levels of IL-6 after a predetermined timepoint after first administration of an IL-1beta antagonist or 3 month after first administration of canakinumab is below 1.15 mg/L or below 2 mg/L.

18. The method according to any of the preceding embodiments, wherein said patient previously has suffered a CV event.

19. The method according to any of the preceding embodiments, wherein said patient previously has suffered a myocardial infarction.

20. A method for reducing risk of progression of OA and/or reducing adverse events associated with OA in a patient, comprising administering an IL-1beta antagonist, wherein said patient has a high sensitivity C-reactive protein (hsCRP) level of ≥2 mg/L assessed before first administration of said IL-1beta antagonist.

21. The method according to embodiment 20, wherein the IL-1beta antagonist is canakinumab.

22. The method of embodiment 20 or 21, comprising administering 150 mg to 300 mg canakinumab.

23. The method according to any of embodiments 20-22, comprising administering 150 mg to 300 mg canakinumab approximately every 3 months.

24. The method according to any of embodiments 20-23, wherein the documented OA has been assessed using X-ray and/or MRI.

25. The method according to any of embodiments 20-24, wherein the symptomatic evidence of OA is pain and/or impaired function.

26. The method according to any of embodiments 20-25, wherein the patient is not eligible for surgery.

27. The method according to any of embodiments 20-26, wherein the patient is not responsive to NSAIDs.

28. The method according to any of embodiments 20-27, wherein said patient previously has suffered a CV event.

29. The method according to any of embodiments 20-28, wherein said patient previously has suffered a myocardial infarction.

30. The method according to any of the preceding embodiments, wherein said total joint replacement can be total knee replacement or total hip replacement.

31. The method according to any of the preceding embodiments, wherein the patient suffers from shoulder OA, hand OA, or spondylarthrosis (degenerative spinal joint disease).

32. The method according to any of the preceding embodiments, where the total joint replacement can be total shoulder replacement.

33. The method according to any of embodiments 1-2 and 7-9, wherein the predetermined time point is 2 weeks up to 6 months.

34. The method according to any of embodiments 1-2 and 7-9, wherein the predetermined time point is 4 weeks to 12 weeks.

Further features and advantages of the disclosure will become apparent from the following detailed description of the invention.

### BRIEF DESCRIPTION OF THE DRAWINGS

FIG. 1 is a graphical representation showing the time for an OA related adverse event in patients with OA in their medical history as a function of canakinumab dosing at several levels versus placebo.

FIG. 2 is a graphical representation of the time to a hip or knee replacement in patients with OA after administration of canakinumab versus placebo.

FIG. 3 is a graphical representation of the risk of an OA related adverse event in groups stratified by hsCRP concentration.

FIG. 4 is a graphical representation of the risk of a total joint replacement (TJR) in patients with a history of OA in groups stratified by hsCRP concentration.

### DETAILED DESCRIPTION OF THE DISCLOSURE

The present invention provides methods of preventing or reducing disease progression of OA, including the need of joint replacement in OA patients; and/or preventing or reducing AEs associated with OA, by administering to such patients an IL-1 beta anatagonist, such as canakinumab.

Canakinumab (international nonproprietary name (INN) number 8836) is disclosed in WO02/16436 which is hereby incorporated by reference in its entirety. Canakinumab is a fully human monoclonal anti-human IL-1β antibody of the IgG1/k isotype, being developed for the treatment of IL-1β driven inflammatory diseases. It is designed to bind to human IL-1β, and thereby blocking the interaction of the cytokine with its receptors. The antagonism of the IL-1β mediated inflammation using canakinumab in lowering high sensitivity C-reactive protein (hsCRP) and other inflammatory marker levels has shown an acute phase response in patients with Cryopyrin-Associated Periodic Syndrome (CAPS) and rheumatoid arthritis. This evidence has been replicated in patients with type 2 diabetes mellitus (T2DM) using canakinumab and with other IL-1β antibody therapies in development, although in T2DM reduction in hsCRP levels did not translate to increased efficaciousness over standard of care treatment. IL-1β inhibition over a longer period of time, thereby inhibiting a major inflammatory pathway, will have unforeseen effects, which may be advantageous or not, therefore necessitating a large, randomized, placebo-controlled clinical trial monitoring multiple parameters.

The inventors have now found that treatment with canakinumab significantly reduces the risk of osteoarthritis, related conditions and side effects. Pro-inflammatory cytokines are critical mediators of the disturbed metabolism and enhanced catabolism of joint tissue involved in OA. IL-1p, TNF and IL-6 seem to be the main pro-inflammatory and pro-catabolic cytokines in OA driving the inflammatory cascade, although also IL-15, IL-17, IL-18, IL-21, leukemia inhibitory factor (LIF) and chemokines are implicated. IL-1β & TNF are produced by chondrocytes, mononuclear cells, osteoblasts and synovial tissues. The activation of cells by IL-1beta is mediated solely by binding to its specific cell surface receptor, IL-1RI. Levels of both IL-1β and TNF are elevated in the synovial fluid, synovial membrane, subchondral bone and cartilage. Furthermore, IL-1β and TNF can act independently or in concert with other cytokines to initiate and propagate inflammation. IL-1β is up-regulate pro-nociceptive mediators (i.e., NGF) resulting in increased pain. Furthermore, IL-1β & TNF stimulate chondrocytes to release several protcolytic enzymes: MMPs: MMPI (interstitial collagenase), MMP3 (stromelysin 1) and MMP13 (collagenase 3).

In one embodiment, any method of the invention comprises administering about 50, 150, 175, 200, 225, 250, 275, 300 mg or any combination thereof of canakinumab.

One embodiment of any method of the invention comprises administering 150 mg canakinumab or 300 mg canakinumab. Another embodiment of any method of the invention comprises administering 150 mg canakinumab. Yet another embodiment comprises administering 225 mg canakinumab. In other embodiments, 50 or 200 mg or canakinumab is administered.

In one embodiment of any method of the invention the reduced level of hsCRP assessed approximately 3 months after first administration of canakinumab is <1.9, <1.8, <1.7, <1.6, <1.5, <1.4, <1.3, <1.2, <1.1, <1.0, <0.9, <0.8, <0.7, <0.6, or <0.5 mg/L. In one embodiment, the reduced level of hsCRP assessed approximately 3 months after first administration of canakinumab is <1.0 mg/L. In another embodiment, the reduced level of hsCRP assessed approximately 3 months after first administration of canakinumab is <2 mg/L. In yet another embodiment, the reduced level of hsCRP is less than or equal to 3 mg/L.

In a further aspect of any method of the disclosure, an initial dose of 150 mg canakinumab is administered to a patient that has suffered and results in a response, i.e. a reduction of hsCRP level in said patient. However, the reduced hsCRP level assessed at least three months after the initial administration of canakinumab is not below 2 mg/L and, instead of stopping the treatment for said patient, a further initial dose of canakinumab is being administered. If the hsCRP level assessed after at least three months after the further initial dose is below 2 mg/L said patient will continue with the treatment and receive subsequent doses of 150 mg or preferably 300 mg canakinumab about every 3 months.

In another aspect of any method of the disclosure, after the initial dose of canakinumab, such as 50 mg, 150, mg, 200, mg, 225 mg or 300 mg, the levels of a relevant biomarker such as IL-6 or hsCRP is measured after a predetermined time, preferably 3 months from the initial dose. Thereafter, the biomarker is measured again after a second predetermined period, preferably 6 months from the initial dose. A second dose may then be administered, such as 50 mg, 150, mg, 200, mg, 225 mg or 300 mg of canakinumab to the patient in response to the measured level of the biomarker.

In one embodiment the method of the invention optionally further comprises administering the patient an additional dose of 300 mg of canakinumab about two weeks (+/−3 days) from initial administration of canakinumab.

Canakinumab can be administered subcutaneously or intravenously. Canakinumab can be administered in a reconstituted formulation comprising canakinumab at a concentration of 50-200 mg/ml, 50-300 mM sucrose, 10-50 mM histidine, and 0.01-0.1% surfactant and wherein the pH of the formulation is 5.5-7.0. Canakinumab can be administered in a reconstituted formulation comprising canakinumab at a concentration of 50-200 mg/ml, 270 mM sucrose, 30 mM histidine and 0.06% polysorbate 20 or 80, wherein the pH of the formulation is 6.5.

Canakinumab can also be administered in a liquid formulation comprising canakinumab at a concentration of 50-300 mg/ml, a buffer system selected from the group consisting of citrate, histidine and sodium succinate, a stabilizer selected from the group consisting of sucrose, mannitol, sorbitol, arginine hydrochloride, and a surfactant and wherein the pH of the formulation is 5.5-7.0. Canakinumab can also be administered in a liquid formulation comprising canakinumab at a concentration of 50-300 mg/ml, 50-300 mM mannitol, 10-50 mM histidine and 0.01-0.1% surfactant, and wherein the pH of the formulation is 5.5-7.0. Canakinumab can also be administered in a liquid formulation comprising canakinumab at a concentration of 50-300 mg/ml, 270 mM mannitol, 20 mM histidine and 0.04% polysorbate 20 or 80, wherein the pH of the formulation is 6.5.

When administered subcutaneously, canakinumab can be administered to the patient in a liquid form contained in a prefilled syringe, autoinjector, or as a lyophilized form for reconstitution.

In other embodiments of any method according to the invention, a biomarker other than hsCRP such as IL-6 can be utilized to determine the response to canakinumab.

Other embodiments of the invention include the use of canakinumab according to any of the described uses or methods herein.

### General:

All patents, published patent applications, publications, references and other material referred to herein are incorporated by reference herein in their entirety.

As used herein, the term “comprising” encompasses “including” as well as “consisting,” e.g. a composition “comprising” X may consist exclusively of X or may include something additional, e.g., X+Y.

As used herein, the term “administering” in relation to a compound, e.g., canakinumab or standard of care agent, is used to refer to delivery of that compound by any route of delivery.

As used herein, the term “about” in relation to a numerical value x means, for example, +/−10%.

As used herein, the word “substantially” does not exclude “completely.” e.g., a composition which is “substantially free” from Y may be completely free from Y. Where necessary, the word “substantially” may be omitted from the definition of the disclosure.

As used herein, in one embodiment the term “3 months” includes a time period that extends one week before and one week after the 3 months (3 months+/−1 week). In another embodiment the term “approximately 3 months” includes a time period of 90 days+/−15 days or 90 days+/−10 days.

The term “biomarker”, as used herein, refers generally to a molecule, i.e., a gene (or nucleic acid encoding said gene), protein, the expression of which in a biological sample from a patient can be detected by standard methods in the art, and is predictive or denotes a condition of the patient from which it was obtained. According to the invention, exemplary biomarkers include but are not limited to hsCRP and IL-6.

As used herein, the term “assaying” is used to refer to the act of detecting, identifying, screening, or determining, which act may be performed by any conventional means. For example, a sample may be assayed for the presence of a particular marker by using an ELISA assay, a Northern blot, imaging, etc. to detect whether that marker is present in the sample.

As used herein, the terms “C-reactive protein” and “CRP” refers to serum C-reactive protein, which is used as an indicator of the acute phase response to inflammation. As used herein, the term “hsCRP” refers to the level of CRP in the blood as measured by high sensitivity CRP testing. The level of CRP or hsCRP in plasma may be given in any concentration, e.g., mg/dl, mg/L, nmol/L. Levels of CRP or hsCRP may be measured by a variety of well-known methods, e.g., radial immunodiffusion, electroimmunoassay, immunoturbidimetry, ELISA, turbidimetric methods, fluorescence polarization immunoassay, and laser nephelometry.

Testing for CRP may employ a standard CRP test or a high sensitivity CRP (hsCRP) test (i.e., a high sensitivity test that is capable of measuring low levels of CRP in a sample, e.g., using laser nephelometry). Kits for detecting levels of CRP or hsCRP may be purchased from various companies, e.g., Calbiotech, Cayman Chemical, Roche Diagnostics Corporation, Abazyme, DADE Behring, Abnova Corporation, Aniara Corporation, Bio-Quant Inc., Siemens Healthcare Diagnostics, etc.

As used herein, the term “patient” and “subject” are used interchangeably.

Other features, objects, and advantages of the invention will be apparent from the description and drawings, and from the claims.

As used herein, the term “osteoarthritis” and “osteoarthropathies” are used interchangeable and encompass a broad array of conditions, such as spinal OA, related spinal degenerative diseases, as well as upper and lower limb OAs. Non-limiting examples are included in the following table:

TABLE 1
NON-LIMITING LIST OF OA TYPES

| Preferred terms used in medical | | Preferred terms used |
|---|---|---|
| history reporting | | in AE reporting |
| Ankle OA | Gonarthrosis | Lumbo-sacral |
| | | spondylosis |
| Arthrosis | Lumbar-sacral | Spinal OA |
| | spondylosis | |
| Ankylosing vertebral | Nodal osteoarthritis | OA knee |
| hyperostosis | | |
| Arthrosis | OA hip | Gonarthrosis |
| deformans | | |
| Arthrosis | Omarthrosis | OA knees aggravated |
| multiple | | |
| Coxarthrosis | OA knees | Coxarthrosis |
| Hyperostotic | OA shoulders | OA aggravated |
| spondylosis | | |
| Hand OA | Osteo-arthritis of | Hip arthrosis |
| | neck | |
| OA of the | OA | Coxarthrosis |
| cervical spine | | |
| Generalized OA | OA generalised | Wrist OA |
| Spondylosis | OA knee | Hand OA |
| Cervical spine | OA knees | OA of the lumbar |
| degeneration | | spine |
| Lumbar spine | OA spinal | OA of the cervical |
| degeneration | | spine |
| Cervical | OA of the cervical | Omarthrosis |
| spondylosis | spine | |
| Lumbar | Osteoarthritis of | Ankle OA |
| spondylosis | lumbar spine | |
| Degenerative | OA of thoracic spine | Lumbar spine |
| joint disease | | degeneration |
| Knee OA | OA shoulders | Cervical spine |
| | | degeneralion |
| Degenerative arthritis | OA spinal | Erosive arthritis |
| peripheral joint | | |
| Degenerative | OA generalised | Elbow OA |
| arthritis spine | | |
| OA | Shoulder OA | Spondylosis |
| Elbow OA | Spinal OA | Thumb OA |
| Finger OA | Spondylosis | Foot OA |
| Shoulder OA | Thoracic spondylosis | Knee OA |
| Foot OA | Thumb OA | OA |
| Forrestier | Toe OA | Shoulder OA |
| disease | | |
| Generalized | Wrist osteoarthritis | OA aggravated |
| osteoarthritis | | |
| | | OA hip |
| | | Spondylosis |
| | | aggravated |
| | | Toe OA |

As used herein, canakinumab is defined under INN number 8836 and has the following sequence:

Light chain
SEQ ID NO: 17

| | |
|---|---|
| 1 | EIVLTQSPDF QSVTPKEKVT ITCRASQSIG SSLHWYQQKP |
| | DQSPKLLIKY ASQSFSGVPS |
| 61 | RFSGSGSGTD FTLTINSLEA EDAAAYYCHQ SSSLPFTFGP |
| | GTKVDIKRTV AAPSVFIFPP |
| 121 | SDEQLKSGTA SVVCLLNNFY PREAKVQWKV DNALQSGNSQ |
| | ESVTEQDSKD STYSLSSTLT |
| 181 | LSKADYEKHK VYACEVTHQG LSSPVTKSFN RGEC* |

Heavy chain:
SEQ ID NO: 18

| | |
|---|---|
| 1 | QVQLVESGGG VVQPGRSLRL SCAASGFTFS VYGMNWVRQA |
| | PGKGLEWVAI IWYDGDNQYY |
| 61 | ADSVKGRFTI SRDNSKNTLY LQMNGLRAED TAVYYCARDL |
| | RTGPFDYWGQ GTLVTVSSAS |
| 121 | TKGPSVFPLA PSSKSTSGGT AALGCLVKDY FPEPVTVSWN |
| | SGALTSGVHT FPAVLQSSGL |
| 181 | YSLSSVVTVP SSSLGTQTYI CNVNHKPSNT KVDKRVEPKS |
| | CDKTHTCPPC PAPELLGGPS |
| 241 | VFLFPPKPKD TLMISRTPEV TCVVVDVSHE DPEVKFNWYV |
| | DGVEVHNAKT KPREEQYNST |
| 301 | YRVVSVLTVL HQDWLNGKEY KCKVSNKALP APIEKTISKA |
| | KGQPREPQVY TLPPSREEMT |
| 361 | KNQVSLTCLV KGFYPSDIAV EWESNGQPEN NYKTTPPVLD |
| | SDGSFFLYSK LTVDKSPWQQ |
| 421 | GNVFSCSVMH EALHNHYTQK SLSLSPGK* |

An antibody, as used herein, refers to an antibody having the natural biological form of an antibody. Such an antibody is a glycoprotein and consists of four polypeptides—two identical heavy chains and two identical light chains, joined to form a “Y”-shaped molecule. Each heavy chain is comprised of a heavy chain variable region (VH) and a heavy chain constant region. The heavy chain constant region is comprised of three or four constant domains (CH1, CH2, CH3, and CH4, depending on the antibody class or isotype). Each light chain is comprised of a light chain variable region (VL) and a light chain constant region, which has one domain. CL. Papain, a proteolytic enzyme, splits the “uY” shape into three separate molecules, two so called “Fab” fragments (Fab=fragment antigen binding), and one so called “Fc” fragment (Fc=fragment crystallizable). A Fab fragment consists of the entire light chain and part of the heavy chain. The VL and VH regions are located at the tips of the “Y”-shaped antibody molecule. The VL and VH each have three complementarity-determining regions (CDRs).

By “IL-1β binding antibody” is meant any antibody capable of binding to the IL-1β specifically and consequently inhibiting or modulating the binding of IL-1β to its receptor and further consequently inhibiting IL-1β function. Preferably an IL-1β binding antibody does not bind to IL-1α.

Preferably an IL-1β binding antibody includes:

(1) An antibody comprising three VL CDRs having the amino acid sequences RASQSIGSSLH (SEQ ID NO: 1), ASQSFS (SEQ ID NO: 2), and HQSSSLP (SEQ ID NO: 3) and three VH CDRs having the amino acid sequences VYGMN (SEQ ID NO: 5), IIWYDGDNQYYADSVKG (SEQ ID NO: 6), and DLRTGP (SEQ ID NO: 7):

(2) An antibody comprising three VL CDRs having the amino acid sequences RASQDISNYLS (SEQ ID NO: 9), YTSKLHS (SEQ ID NO: 10), and LQGKMLPWT (SEQ ID NO: 11), and three VH CDRs having the amino acid sequences TSGMGVG (SEQ ID NO: 13), HIWWDGDESYNPSLK (SEQ ID NO: 14), and NRYDPPWFVD (SEQ ID NO: 15); and

(3) An antibody comprising the six CDRs as described in either (1) or (2), wherein one or more of the CDR sequences, preferably at most two of the CDRs, preferably only one of the CDRs, differ by one amino acid from the corresponding sequences described in either (1) or (2), respectively.

Preferably an IL-1β binding antibody includes:

(1) An antibody comprising three VL CDRs having the amino acid sequences RASQSIGSSLH (SEQ ID NO: 1), ASQSFS (SEQ ID NO: 2), and HQSSSLP (SEQ ID NO: 3) and comprising the VH having the amino acid sequence specified in SEQ ID NO: 8:

(2) An antibody comprising the VL having the amino acid sequence specified in SEQ ID NO: 4 and comprising three VH CDRs having the amino acid sequences VYGMN (SEQ ID NO: 5), IIWYDGDNQYYADSVKG (SEQ ID NO: 6), and DLRTGP (SEQ ID NO: 7):

(3) An antibody comprising three VL CDRs having the amino acid sequences RASQDISNYLS (SEQ ID NO: 9), YTSKLHS (SEQ ID NO: 10), and LQGKMLPWT (SEQ ID NO: 11), and comprising the VH having the amino acid sequences specified in SEQ ID NO: 16;

(4) An antibody comprising the VL having the amino acid specified in SEQ ID NO: 12, and comprising three VH CDRs having the amino acid sequences TSGMGVG (SEQ ID NO: 13), HIWWDGDESYNPSLK (SEQ ID NO: 14), and NRYDPPWFVD (SEQ ID NO: 15);

(5) An antibody comprising three VL CDRs and the VH sequence as described in either (1) or (3), wherein one or more of the VL CDR sequences, preferably at most two of the CDRs, preferably only one of the CDRs, differ by one amino acid from the corresponding sequences described in (1) or (3), respectively, and wherein the VH sequence is at least 90% identical to the corresponding sequence described in (1) or (3), respectively; and

(6) An antibody comprising the VL sequence and three VH CDRs as described in either (2) or (4), wherein the VL sequence is at least 90% identical to the corresponding sequence described in (2) or (4), respectively, and wherein one or more of the VH CDR sequences, preferably at most two of the CDRs, preferably only one of the CDRs, differ by one amino acid from the corresponding sequences described in (2) or (4), respectively.

Preferably an IL-1β binding antibody includes:
(1) An antibody comprising the VL having the amino acid sequence specified in SEQ ID NO: 4 and comprising the VH having the amino acid sequence specified in SEQ ID NO: 8;
(2) An antibody comprising the VL having the amino acid specified in SEQ ID NO: 12, and comprising the VH having the amino acid sequences specified in SEQ ID NO: 16; and
(3) An antibody described in either (1) or (2), wherein the constant region of the heavy chain, the constant region of the light chain or both has been changed to a different isotype as compared to canakinumab or gevokizumab.

Preferably an IL-1β binding antibody includes Canakinumab (SEQ ID NO:17 and 18).

An IL-1β binding antibody as defined above has substantially identical or identical CDR sequences as those of canakinumab. It thus binds to the same epitope on IL-1β and has similar binding affinity as canakinumab or gevokizumab. The clinical relevant doses and dosing regimens that have been established for canakinumab as therapeutically efficacious in the treatment of OA would be applicable to other IL-1β binding antibodies.

Additionally or alternatively, an IL-1β antibody refers to an antibody that is capable of binding to IL-1β specifically with affinity in the similar range as canakinumab. The Kd for canakinumab in WO2007/050607 is referenced with 30.5 pM. Thus affinity in the similar range refers to between about 0.05 pM to 300 pM, preferably 0.1 pM to 100 pM. It does not prevent IL-1β from binding to the receptor but prevent recetor activation. Preferably an IL-1β antibody has the binding affinity in the similar range as canakinumab, preferably in the range of 1 pM to 300 pM, preferably in the range of 10 pM to 100 pM, wherein preferably said antibody directly inhibits binding.

As used herein, the term “functional fragment” of an antibody as used herein, refers to portions or fragments of an antibody that retain the ability to specifically bind to an antigen (e.g., IL-1β). Examples of binding fragments encompassed within the term “functional fragment” of an antibody include single chain Fv (scFv), a Fab fragment, a monovalent fragment consisting of the V_{L}, V_{H}, CL and CH1 domains; a F(ab)2 fragment, a bivalent fragment comprising two Fab fragments linked by a disulfide bridge at the hinge region; a Fd fragment consisting of the V_{H} and CH1 domains; a Fv fragment consisting of the V_{L} and V_{H} domains of a single arm of an antibody; a dAb fragment (Ward et al., 1989), which consists of a V_{H} domain; and an isolated complementarity determining region (CDR); and one or more CDRs arranged on peptide scaffolds that can be smaller, larger, or fold differently to a typical antibody.

The term “functional fragment” might also refer to one of the following:
bispecific single chain Fv dimers (PCT/US92/09965)
“diabodies” or “triabodies”, multivalent or multispecific fragments constructed by gene fusion (Tomlinson I & Hollinger P (2000) Methods Enzymol. 326: 461-79; WO94113804; Holliger P et al., (1993) Proc. Natl. Acad. Sci. USA, 90: 6444-48)
scFv genetically fused to the same or a different antibody (Coloma MJ & Morrison SL (1997) Nature Biotechnology, 15(2): 159-163)
scFv, diabody or domain antibody fused to an Fc region
scFv fused to the same or a different antibody
Fv, scFv or diabody molecules may be stabilized by the incorporation of disulphide bridges linking the VH and VL domains (Reiter, Y. et al, (1996) Nature Biotech, 14, 1239-1245).
Minibodies comprising a scFv joined to a CH3 domain may also be made (Hu, S. et al, (1996) Cancer Res., 56, 3055-3061).
Other examples of binding fragments are Fab′, which differs from Fab fragments by the addition of a few residues at the carboxyl terminus of the heavy chain CH1 domain, including one or more cysteines from the antibody hinge region, and Fab′-SH, which is a Fab′ fragment in which the cysteine residue(s) of the constant domains bear a free thiol group

Typically and preferably an functional fragment of an IL-1β binding antibody is a portion or a fragment of an “IL-1β binding antibody” as defined above.

Other features, objects, and advantages of the invention will be apparent from the description and drawings, and from the claims.

The following Examples illustrate the invention described above; they are not, however, intended to limit the scope of the invention in any way.

### Example 1: The Cantos Trial

Data generated from the CANTOS trial is disclosed in WO2013/049278, the entire contents of which are hereby incorporated by reference. CANTOS was a randomized, double-blind, placebo-controlled, event-driven trial, designed to evaluate whether the administration of quarterly subcutaneous canakinumab can prevent recurrent cardiovascular events among stable post-myocardial infarction patients with elevated hsCRP. The enrolled 10,061 patients with myocardial infarction and inflammatory atherosclerosis had high sensitivity C-reactive protein (hsCRP) of ≥2 mg/L. Three escalating canakinumab doses (50 mg, 150 mg, and 300 mg given subcutaneously every 3 months) were compared to placebo.

The following details the setup and results of the CANTOS trial, identified as NTC01327846, the contents of which are hereby incorporated by reference in their entirety.

A Randomized, Double-Blind, Placebo-Controlled, Event-Driven Trial of Quarterly Subcutaneous Canakinumab in the Prevention of Recurrent Cardiovascular Events Among Stable Post-Myocardial Infarction Patients with Elevated hsCRP.

This study was designed as a multi-center, randomized, parallel group, placebo-controlled, double-blind, event-driven trial to provide definitive evidence on the effects of canakinumab on cardiovascular adverse events in patients with recent MI and elevated inflammatory burden as evidenced by elevated hsCRP. This study design was the most robust clinical trial design to test the hypothesis that anti-inflammatory treatment with canakinumab reduce major adverse cardiovascular events.

### Rationale of Study Design

Trial Population. Patients were eligible for enrollment if they had a prior history of myocardial infarction and had blood levels of hsCRP of 2 mg/L or greater despite use of aggressive secondary prevention strategies. The trial excluded from enrollment those with a history of chronic or recurrent infection, prior malignancy other than basal cell skin carcinoma, suspected or known immunocompromised state, a history of or high risk for tuberculosis or HIV-related disease, or ongoing use of other systemic anti-inflammatory treatments.

Inclusion Criteria

Patients eligible for inclusion in the study had to fulfill all of the following criteria:

1. Written informed consent obtained before any assessment performed.
2. Male, or Female of non-child-bearing potential
3. Age ≥18 years at Visit 1.
4. Documented spontaneous MI (diagnosed according to the universal Mil criteria with or without evidence of ST segment elevation) at least 30 days before randomization.
Diagnosis of the qualifying MI should be based on medical history of clinical symptoms consistent with myocardial ischemia associated with elevation of cardiac biomarkers above the 99th percentile of the upper reference limit (preferably troponin) OR development of new pathological Q waves regardless of symptoms. For details, refer to the Universal Definition of MI.
   a. Acute MI (hospitalization records): requires documentation of a rise and/or fall of cardiac biomarkers (preferably troponin) with at least one value above the 99th percentile of the upper reference limit (URL) or above criteria diagnostic for MI and evidence of myocardial ischemia as demonstrated by at least one of the following:
   i. Symptoms of ischemia
   ii. ECG changes indicative of new ischemia (new ST-T changes or new LBBB)
   iii. Development of pathologic Q waves
   iv. Imaging evidence of new loss of viable myocardium or new regional wall motion abnormality
   b. Prior MI (no hospital records for acute event available): requires documentation of any one of the following:
   i. Development of pathological Q waves, with or without symptoms
   ii. Imaging evidence of a region of loss of viable myocardium that is thinned and fails to contract, in the absence of a non-ischemic cause
   iii. Pathologic findings of a healed or healing MI
Patients with MI resulting from PCI or CABG were not eligible
   5. Have an hsCRP ≥2 mg/L (collected less than 60 days prior to Visit 2 and performed at the central laboratory, which is a minimum of 28 days after qualifying MI or after any PCI performed separately from qualifying MI) on stable (at least 4 weeks) long term (cardiovascular) medications (standard of care).

Randomization. Patients were initially randomized to canakinumab 150 mg, canakinumab 300 mg, or placebo in a 1:1:1 ratio. After the enrollment of 741 participants, a 50 mg dose was added at regulatory request, with the randomization ratio adjusted accordingly; we sought to achieve a final randomization ratio of 1.5:1:1:1. All study-drug doses and placebo were administered subcutaneously once every three months; for the 300 mg dose, the regimen was 300 mg every two weeks for the first two doses, then once every three months.

Randomization was performed with the use of a centralized computer system, with stratification by time since index myocardial infarction and by trial part (before versus after inclusion of the 50 mg dose).

End Points. The primary efficacy end point was time to first occurrence of nonfatal myocardial infarction, any nonfatal stroke, or cardiovascular death. The trial had two key secondary efficacy end points. The first key secondary end point included the components of the primary end point as well as hospitalization for unstable angina requiring urgent revascularization. The two other pre-specified secondary end points were all-cause mortality and the composite of nonfatal myocardial infarction, any nonfatal stroke, or all-cause mortality. All components of these end points were adjudicated by an end point adjudication committee, with members masked to study-drug assignment.

Statistical Analysis. Distributions of percent change from baseline in hsCRP and lipid levels were compared between placebo and each canakinumab group at intervals up to 48 months. Similar comparisons were made for IL-6 up to 12 months. Log-rank tests and Cox proportional-hazards models, stratified by time since index myocardial infarction and trial part, were used to analyze the pre-specified primary and key secondary cardiovascular outcomes that occurred during trial follow-up according to the intention-to-treat principle. Formal evaluation of significance for individual doses, adjusted for multiplicity, followed a closed testing procedure. Based on the closed testing procedure, and using the pre-specified allocation of alpha error, the two-sided P value thresholds for statistical significance for the primary end point were 0.01058 for the test of the 300 mg dose of canakinumab versus placebo and 0.02115 for the tests of the other two doses versus placebo. The closed testing procedure also specified that formal significance testing for the key secondary end points would be performed for any given dose only if the significance threshold for the primary end point for that dose had been met.

While the primary analysis strategy was based on pair-wise comparisons of individual dose groups to the placebo group, comparisons were also made between incidence rates on placebo and incidence rates across ascending canakinumab doses (using scores of 0, 1, 3, and 6 proportional to doses in a trend analysis) and for the combined active canakinumab treatment groups versus placebo. In addition, on-treatment analyses were performed with follow-up for each patient censored 119 days after the last study injection received. The significance thresholds for these tests were not adjusted for multiplicity. Similar analyses were used for adverse events. All P values are two-sided and all confidence intervals computed at the 95% level.

Patients. Trial enrollment began in April 2011 and was completed in March 2014; the last trial visit was in June 2017. Of 17,482 post-infarction patients who underwent screening in the central laboratory, 10,061 (57.6%) were correctly randomized and received at least one dose of trial medication. The most common reasons for exclusion were hsCRP less than 2 mg/L (46% of excluded subjects), active tuberculosis or tuberculosis risk factors (25.4%), and exclusionary concomitant disorders (9.9%).

The mean age of randomized participants was 61 years, 26% were women, and 40% had diabetes. Most participants had undergone prior revascularization procedures (67% percutanous coronary interventions, 14% coronary bypass surgery). At baseline, anti-thrombotic therapy was taken by 95%, lipid-lowering therapy by 93%, anti-ischemia agents by 91%, and inhibitors of the renin-angiotensin system by 79%. The median hsCRP at entry was 4.2 mg/L and the median LDL cholesterol was 82 mg/dL.

Effects on inflammatory biomarkers and lipid levels. Compared to placebo, at 48 months, hsCRP was reduced by 26%, 37%, and 41% in the canakinumab 50 mg, 150 mg, and 300 mg groups, respectively (all P-values <0.001 in comparisons of the median percent change on canakinumab to the median percent change on placebo). Similar effects were observed for IL-6 (measured up to 12 months). By contrast, canakinumab use resulted in no reduction in LDL cholesterol or HDL cholesterol, and a 4 to 5% median increase in triglycerides.

Follow-up and Effects on Clinical End Points: By the end of follow-up, 18.1% of patients in the placebo group had discontinued study drug, as compared to 18.7% of patients in the combined canakinumab groups. At a median follow-up of 3.7 years, the incidence rates for the primary end (which included nonfatal myocardial infarction, nonfatal stroke, or cardiovascular death) in the placebo, 50 mg, 150 mg, and 300 mg groups were 4.50, 4.11, 3.86, and 3.90 per 100 person-years, respectively. No significant effect was observed for the primary end point in the canakinumab 50 mg dose group compared to placebo (hazard ratio [HR] 0.93, P=0.30). By contrast, a statistically significant effect for the primary end point was observed in the canakinumab 150 mg dose group (HR 0.85, P=0.02075, threshold P value 0.02115). In the canakinumab 300 mg dose group, the hazard ratio was similar but the P value did not meet the pre-specified significance threshold (HR 0.86, P=0.0314, threshold P value 0.01058). The P value for trend across the active-dose groups compared to placebo was 0.020, and the P value for comparison of all doses combined versus placebo was 0.015 (both results not adjusted for multiple testing). Additionally, a subgroup of patients showing greater reductions in their hsCRP levels after treatment with canakinumab after 3 months show a statistically significant greater risk reduction in MACE compared to the overall treatment population. Patients responding with reductions in their hsCRP levels to <1.8 mg/L receiving 150 mg and 300 mg canakinumab, respectively, showed 24% and 22% relative risk reduction in MACE, respectively, based on causal inference analysis assuming exponential survival distribution, estimates based on 500 bootstrap samples. Patients responding with reductions in their hsCRP levels to <1.5 mg/L receiving 150 mg and 300 mg canakinumab, respectively, showed a 26% and 27% relative risk reduction in MACE, respectively, based on causal inference analysis assuming exponential survival distribution, estimates based on 500 bootstrap samples.

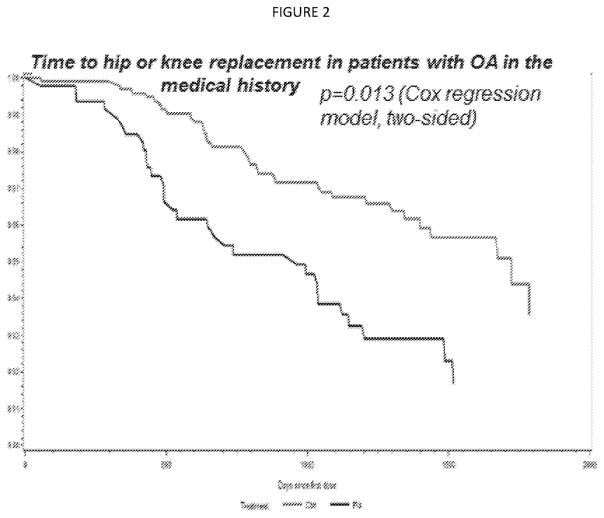

For the key secondary cardiovascular end point (which included the components of the primary end point plus hospitalization for unstable angina requiring urgent revascularization), incidence rates in the placebo, 50 mg, 150 mg, and 300 mg groups were 5.13, 4.56, 4.29, and 4.25 per 100 person-years, respectively (Table 2). For the canakinumab 150 mg dose (for which the P value met the significance threshold for the primary end point), the hazard ratio for the secondary cardiovascular endpoint was 0.83 (P=0.00525, threshold P value 0.00529) (FIG. 2D). According to the closed testing procedure, formal significance testing for the pre-specified secondary end point was not performed for the 50 mg and 300 mg doses. The hazard ratios for these doses were 0.90 and 0.83, respectively. The P value for trend across the active-dose groups compared to placebo was 0.003, and the P value for comparison of all doses combined versus placebo was 0.001 (both results not adjusted for multiple testing).

Analyses of the additional secondary end points, and of the components of the primary and secondary end points, were not adjusted for multiple testing. Nominally significant reductions were seen in myocardial infarction for the 150 mg dose of canakinumab; in hospitalization for unstable angina requiring urgent revascularization for the 150 mg and 300 mg doses; and in any coronary revascularization for all three doses. All-cause mortality was neutral in comparisons of all canakinumab doses to placebo (HR 0.94, 95% CI 0.83-1.06, P=0.31). In on-treatment analyses for the primary end point, the observed hazard ratios in the placebo, 50 mg, 150 mg, and 300 mg groups were 1.0, 0.90, 0.83, and 0.79 (P-trend across groups=0.003). In comparable analyses for the key secondary cardiovascular end point, the corresponding hazard ratios were 1.0, 0.88, 0.80, and 0.77 (P-trend across groups <0.001).

Adverse Events and Other Clinical Outcomes. Neutropenia was more common among those allocated to canakinumab and there was a statistically significant increase in fatal events attributed to infection or sepsis when the three canakinumab groups were pooled and compared to placebo (incidence rates 0.31 versus 0.18 per 100 person years, P=0.023). Participants succumbing to infection tended to be older and more likely to have diabetes. Six confirmed cases of tuberculosis occurred in the trial with similar rates in the canakinumab and placebo groups (0.06%); five cases occurred in India and one in Taiwan.

Thrombocytopenia was more common among those allocated to canakinumab, but no difference in hemorrhage was observed. No increase in injection site reactions was observed. Consistent with known effects of IL-1β inhibition, canakinumab resulted in significant reductions in reports of arthritis, gout, and osteoarthritis (discussed in greater detail in Example 2). There was also a significant reduction in cancer mortality with canakinumab.

CANTOS was designed to test directly the inflammatory hypothesis of atherothrombosis. In this trial, among patients with a prior history of myocardial infarction, hsCRP levels and IL-6 levels were significantly reduced by canakinumab, with no reduction in lipid levels. While the 50 mg dose of canakinumab did not have a statistically significant effect on the primary cardiovascular end point compared to placebo, participants in the 150 mg dose group experienced relative hazard reductions of 15% for the primary end point (from 4.50 to 3.86 events per 100 person-years) and 17% for the key secondary cardiovascular end point (from 5.13 to 4.29 events per 100 person-years). The P values for both of these end points met pre-specified multiplicity-adjusted thresholds for statistical significance. Although the hazard reductions for the 300 mg dose group were similar to those for the 150 mg dose group, the prespecified thresholds for statistical significance were not met for this group. Both a pooled analysis of all canakinumab doses and a trend analysis, however, suggested a beneficial effect of canakinumab on cardiovascular outcomes. Specific targeting of IL-1β as a cytokine-based therapy for the secondary prevention of atherosclerotic events rests on several observations. The pro-inflammatory cytokine IL-1β plays multiple roles in atherothrombotic plaque development including induction of procoagulant activity, promotion of monocyte and leucocyte adhesion to vascular endothelial cells, and the growth of vascular smooth muscle cells. In mice, deficiency of IL-1β reduces lesion formation, while in cholesterol-fed pigs, exposure to exogenous IL-1β increases intimal medial thickening. The Nod-like receptor protein 3 (NLRP3) inflammasome activates IL-1p, a process promoted by cholesterol crystals, neutrophil extracellular traps, local hypoxia, and atheroprone flow. This activation of IL-1ß stimulates the downstream IL-6 receptor signaling pathway, implicated by Mendelian randomization studies as a potential causal pathway for atherothrombosis. Most recently, parabiotic mouse studies and studies of clonal hematopoicsis have implicated IL-1β in processes by which bone marrow activation accelerates atherosclerosis. Further, expression of specific inflammasome gene modules impacting IL-1β associates with all-cause mortality and increased atherosclerosis in the elderly.

Although the patients in CANTOS had generally well-controlled levels of LDL cholesterol, placebo event rates were high, with a cumulative incidence of over 20% at five years. Our data thus affirm that statin-treated patients with residual inflammatory risk as assessed by baseline hsCRP greater than 2 mg/L have future event rates at least as high as, if not higher than, statin-treated patients with residual risk due to LDL cholesterol. These two patient groups may differ and may require personalized approaches to treatment. Despite the fact that no reduction in cholesterol levels occurred, the magnitude of effect on cardiovascular events with canakinumab (given every 3 months) was comparable to that associated with monoclonal antibodies targeting PCSK9 (given every 2 to 4 weeks). Yet inhibition of IL-1β is a narrowly focused intervention that represents only one of many potential anti-inflammatory pathways that might serve as targets for atheroprotection. We observed a statistically significant increase in fatal infection and sepsis with canakinumab, as well as a reduction in platelet counts with no increase in bleeding. By contrast, there was a significant reduction in cancer mortality among those allocated to canakinumab, a finding consistent with experimental data relating IL-1 to the progression and invasiveness of certain tumors, in particular lung cancer. There was no significant difference between treatment groups in all-cause mortality. No significant hepatic toxicity was noted. The beneficial effects of canakinumab observed for arthritis, gout, and osteoarthritis are consistent with well-described effects of the IL-1 and IL-6 pathways in these disorders. In conclusion, in CANTOS, patients with a prior history of myocardial infarction and hsCRP levels of 2 mg/L or greater were randomized to one of three doses of canakinumab or placebo. Canakinumab significantly reduced hsCRP levels without reducing LDL cholesterol, HDL cholesterol and triglycerides and the 150 mg dose significantly reduced the incidence of recurrent cardiovascular events whilst having an acceptable levels of side effects.

### Example 2: Canakinumab ((Ilaris®) Prevents Hip and Knee Replacement (THR/TKR) in Patients with OA: Results from the Canakinumab Anti-Inflammatory Thrombosis Outcomes Study (CANTOS) Study

### Background/Purpose:

In OA, there are no therapeutics to prevent disease progression (DMOADs). Canakinumab, a monoclonal antibody targeting interleukin-1β, reduced inflammation and cardiovascular event rates in the CANTOS study. The CANTOS study included a total of 10,061 men and women with a history of myocardial infarction and a high-sensitivity C-reactive protein level of ≥2 mg/L randomized to placebo or one of three doses of canakinumab (50 mg, 150 mg, or 300 mg) given subcutaneously once every 3 months. The median follow-up was 3.7 years.

### Methods:

A post-hoc analysis of the CANTOS data designed to address the effect of canakinumab on the rates of OA-related adverse events (AEs) and serious adverse events (SAEs, as well as total knee replacements (TKR) and total hip replacements (THR) specifically) in all patients and in patients with a medical history of OA. The relationship of OA-related events according to on-treatment concentrations of hsCRP and IL-6 were also investigated. The high level term Osteoarthropath (AP) was used to search the clinical database. A time to event analysis was done for first occurrence of an OAP related AE. The drug treated groups were compared to placebo by two-sided log-rank test. Second, the drug treated groups were pooled, and the time to an OA-related AE, SAE, and TKR/THR was analyzed by Cox proportional hazards regression.

The table below sets forth the analysis:

TABLE 2
Patient distribution by treatment group for All patients and for the subset with osteoarthropathy/no
steoarthropathy/osteoarthritis/spinal osteoarthritis in the medical history (The percentage
indicates the % of the total treatment group (FAS dataset))

| | | | | | | | Combined | | | | | |
|---|---|---|---|---|---|---|---|---|---|---|---|---|
| | CAN | | CAN | | CAN | | CAN | | | | | |
| Dataset | 50 mg | | 150 mg | | 300 mg | | groups | | Placebo | | Total | |
| FAS | 2170 | | 2284 | | 2263 | | 6717 | | 3344 | | 10061 | |
| dataset | | | | | | | | | | | | |
| Safety | 2170 | | 2285 | | 2263 | | 6718 | | 3348 | | 10066 | |
| dataset | | | | | | | | | | | | |
| OAP | 308 | (14.2%) | 371 | (16.2%) | 394 | (17.4%) | 1073 | (16.0%) | 496 | (14.8%) | 1569 | (15.6%) |
| OA | 261 | (12.0%) | 331 | (14.5%) | 343 | (15.2%) | 935 | (13.9%) | 434 | (13.0%) | 1369 | (13.6%) |
| Spinal OA | 66 | (3.0%) | 60 | (2.6%) | 76 | (3.4%) | 202 | (3.0%) | 90 | (2.7%) | 292 | (2.9%) |

CAN = Canakinumab

As shown above, a total of 1569 (15.6%) patients had a medical history of OAP (combined canakinumab groups N=1073, placebo group N=496). A total of 259 (16.5%) OA related AEs, 82 (5.2%) SAEs and 67 (4.3%) TH-RITKR occurred in OAP patients. In the total population there were 52 THR and 47 TKR, corresponding to 0.98% of the total CANTOS population. HsCRP and IL-6 was reduced in a dose-response manner with 300 mg canakinumab resulting in a 46% reduction in hsCRP and IL-6 compared to placebo at 3 months. Table 3 sets forth the results of the reduction in OA and degenerative effects:

TABLE 3
Results of reduction in OA and Degenerative Effects

| | Patients with history of | | | All CANTOS patients | | |
|---|---|---|---|---|---|---|
| | OA at entry N = 1569 | | | N = 10061 | | |
| | Relative risk | | | Relative risk | | |
| AE Type | reduction | | | reduction | | |
| RRR vs. placebo | [95% CI] | | p = | [95% CI] | | p = |
| OA related AEs, pooled doses | 31% | [11%-46%] | 0.003 | 23% | [9%-35%] | 0.002 |
| OA related SAEs, pooled doses | 33% | [0%-62%] | 0.05 | 35% | [7%-55%] | 0.018 |
| THR/TKR, pooled doses | 45% | [12%-66%] | 0.013 | 45% | [18%-63%] | <0.001 |
| THR and TKR, CAN 150 mg | 53% | [7%-76%] | 0.03 | 54% | [17%-74%] | 0.001 |

### Conclusion:

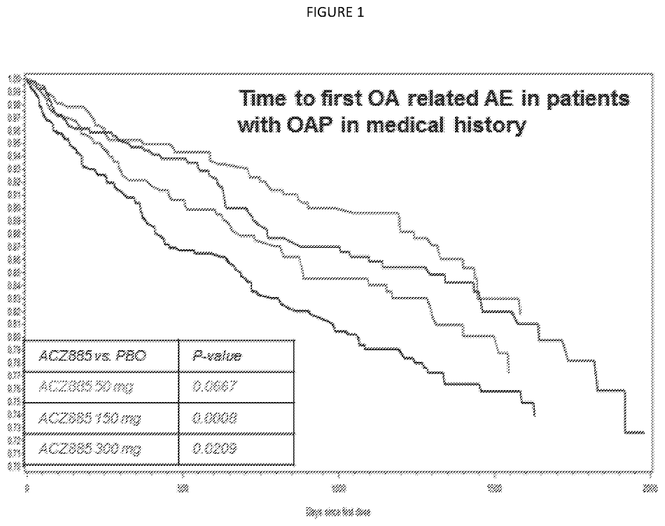

Treatment with canakinumab reduced the risk of worsening of OA (AEs and SAEs) (“RRR”) and significantly reduced the risk of THRs and TKRs in patients with known preexisting OA as well as in the overall CANTOS population, providing evidence of a DMOAD effect of canakinumab in this population. Canakinumab demonstrated a reduction in OAP related AEs, SAEs compared to placebo regardless of having medical history of OA. In the total population within the double blind phase (with a median follow up time of 3.7 years) canakinumab lowers the risk of an OAP related AE by 23% [95% CI; 9%-35%], p=0.002 compared to placebo. The time to first OAP related AE by treatment is presented in FIG. 1 below, which demonstrates significant reduction of AEs over time compared to placebo for 50 mg and 150 mg canakinumab (p-value 0.0033, 0.0016, respectively. For 300 mg canakinumab the p-value was 0.0688.

The results are clear:
There were a total of 123 OA related SAEs in the database
The classification into total hip/knee replacements (THR/TKR) was adjudicated between 2 TMEs, two uncertain cases were adjudicated to: one to replacements, the other as ‘other’ surgery
There were 52 THR and 47 TKR, corresponding to 0.98% of the total CANTOS population
   The results exemplified in FIGS. 1 and 2 show the striking results. As seen in FIG. 1, there is a clear dose-dependent time to the first OA related AE in patients. The time to a first OA increases at the three measured dosages of 50, 150 and 300 mg of canakinumab. As shown in the tables above, in all patients & in patients with OA in the medical history there was a significant 45% relative risk reduction in the pooled canakinumab groups and placebo in time to hip or knee replacement. FIG. 2 shows the average time to a hip or knee replacement in patients with OA. Canakinumab clearly shows a marked improvement versus placebo. Accordingly, canakinumab is very potent in reducing the risk of knee and hip replacements.

### Example 3: OA Related AEs as a Function of hsCRP Levels

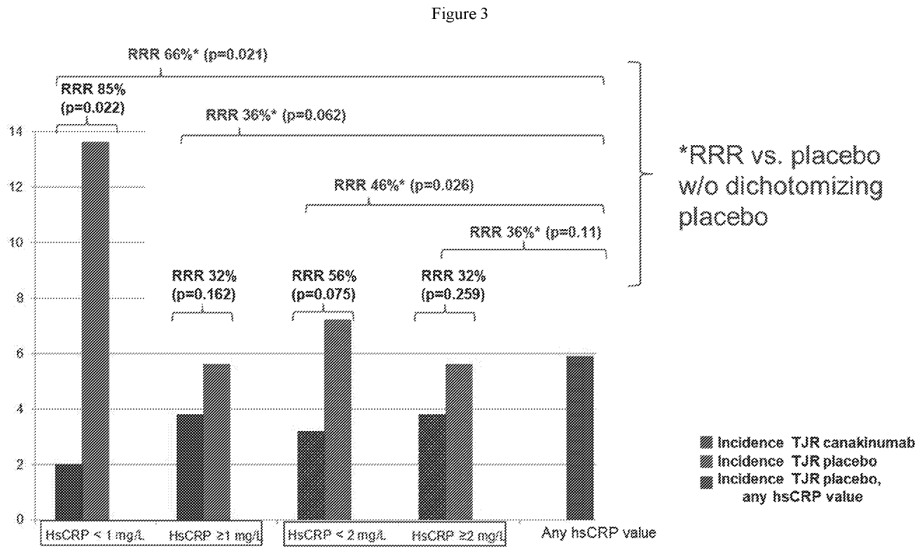

FIG. 3 represents the graphical representation of the risk of an OA related AE in groups stratified by hsCRP concentration. For this table, a total of 259 (16.5%) OA related AEs occurred in patients with OA in the history. Patients were stratified based on the hsCRP level at 3 months <1 mg or ≥1 mg & <2 mg or ≥2 mg and levels correlated to OA related AEs over the study period. It is clear from the graph that there was a higher response rate in patients with lower levels of hsCRP both for a cutoff I and 2 mg/L, regardless if compared to placebo patients with a similar level of hsCRP or any level of hsCRP (without stratifying).

### Example 4: Total Joint Replacement in Patients with OA as a Function of hsCRP Levels

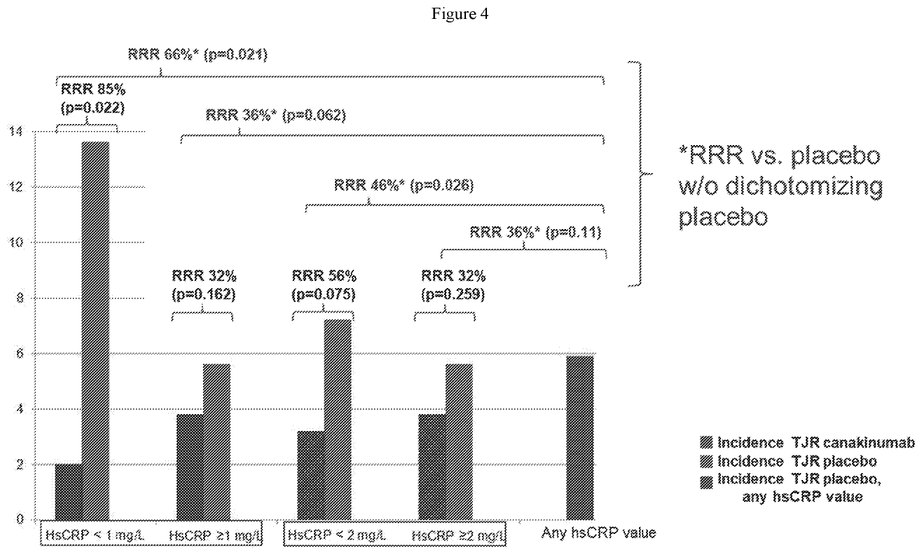

FIG. 4 represents the graphical representation of the total number of joint replacements in patients with a history of OA as a function of hsCRP levels. For this table, a total of 67 (4.3%) THR/TKR occurred in patients with OA in the medical history. Patients were stratified based on the hsCRP level at 3 months <1 mg or ≥1 mg & <2 mg or ≥2 mg and levels correlated to hip/knee replacement (TJR) over the study period. The table clearly shows that there was a higher response rate in patients with a lower levels of hsCRP both for a cutoff I and 2 mg/L.

### Example 5: Confirmatory OA Phase III Study

a. Objective

The objective of this study is to demonstrate that canakinumab reduces structural progression of OA in patients with a high inflammatory burden (hsCRP level of ≥2 mg/L). This study with the results from the CANTOS will be used to support registration canakinumab for the treatment of osteoarthritis in patients with hsCRP ≥2 mg/L at treatment initiation.

b. Patient Population

Adult Patient diagnosed with osteoarthritis who meet the following criteria:
Key inclusion criteria
   1. Age ≥40 years
   2. Body weight >35 or 40 kg, body mass index (BMI)<40 kg/m2.
   3. Diagnosed for knee osteoarthritis based on clinical and radiological criteria of the American College of Rheumatology.
   4. High-sensitivity C-reactive protein (hsCRP) level of ≥2 mg/L
   5. History of knee pain for at least 6 months and on the majority of days (>50%) during the preceding month.
   6. Symptom severity defined by a pain ≥40 mm and ≤90 mm on VAS (100 mm).
   7. Documented need for symptomatic as needed-treatment for OA in the target knee with systemic non-steroidal anti-inflammatory drugs (NSAIDs) and/or other analgesics
   8. WPI<8
Key exclusion criteria
   1. Severe clinical knee malalignment according to the investigator.
   2. Knee prosthesis already implanted (<1 year) or not well-tolerated (contralateral side).
   3. Knee prosthesis already foreseen within the study period (whichever side)
   4. Hip prosthesis recently implanted (<1 year) or foreseen within the study period (whichever side).
   5. Previous osteotomy on the inferior limbs (whichever side).
   6. Surgical operation on the target knee within the 12 months prior to the screening visit or planned during the study.
   7. Arthroscopy of the target knee within the 6 months prior to the screening visit or planned during the study.
   8. Other pathologies affecting the knee.
   9. Any contraindication to MRI including the inability to undergo a knee MRI exam because of inability to fit in the scanner or knee coil.
   10.
      c. Dosing Regimen

The 150 mg s.c. every 3 months dosing regimen of canakinumab is selected as the dosing schedule. This dosing regimen is selected on the basis of the pharmacokinetic (PK) and pharmacodynamics (PD) properties of canakinumab, the observed safety, biomarker and efficacy data from the CANTOS study, and the safety data from completed and ongoing canakinumab studies.

d. Sample Size

Patients will be randomized in a 1:1 ratio to one of the following two treatment arms:
Canakinumab 150 mg s.c. q3 months
Matching Placebo s.c. q3m
   e. Duration of Treatment

Study conduct will be 52/104 weeks

f. Primary Endpoint

This phase III study is designed to demonstrate that canakinumab reduces structural progression of OA. The primary endpoint of the study is Change from baseline in cartilage thickness of the central medial tibiofemoral compartment (cMTFC) assessed by quantitative MRI on the target knee at Week 52.

g. Secondary Endpoints
1. Proportion of OA structural progressors based on cartilage thickness in the central medial tibiofemoral compartment (cMTFC) assessed by quantitative MRI on the target knee at Week 52.
2. Change from baseline in Western Ontario and McMaster Universities Osteoarthritis Index (WOMAC) subscales scores for pain, function, and stiffness at Weeks 24 and Week 52.
3. Change from baseline in pain in the target knee measured with a 100-mm visual analog scale (VAS) at Weeks 24 and 52.
4. Change from baseline in patient global assessment (PGA) of disease activity measured with 100-mm visual analog scale (VAS) at Weeks 24 and 52.
5. Proportion of OMERACT-OARSI responders at Week 52.
   Based on OMERACT-OARSI Initiative: Osteoarthritis Research Society International set of responder criteria for OA clinical trials revisited Pham et al. 2004. A responder is defined according to WOMAC and PGA as a patient who had a high improvement in pain or in function ≥50% and absolute change ≥20 or, improvement in at least 2 of the 3 following:
   Pain ≥20% and absolute change ≥10
   Function ≥20% and absolute change ≥10
   Patient's global assessment ≥20% and absolute change ≥10.
6. Change from baseline in cartilage thickness of the total tibiofemoral compartment (tTFC) of the target knee by quantitative MRI at Week 52.
7. Change from baseline in bone area of the medial femoral condyle surface of the target knee by quantitative MRI at Week 52
8. Change from baseline in bone area of the medial femoral condyle surface of the target knee by quantitative MRI at Week 52.
9. The change from baseline in Joint Space Width (JSW) of the target knee measured by X-Ray at Week 52.
10. The change from baseline in SF36-PCS at Week 24 and Week 52.
11. The change from baseline in SF36-MCS at Week 24 and Week 52.
12. The change in synovitis from MOAKS
13. Pain: analgesic consumption throughout the study over time.

While various specific embodiments are illustrated and described below, it will be appreciated that various changes can be made without departing from the spirit and scope of the disclosure.

1. A method for reducing risk of progression of osteoarthritis (“OA”) and/or reducing adverse events associated with OA in a patient, comprising administering an IL-1beta antagonist, wherein said patient has a high sensitivity C-reactive protein (hsCRP) level of ≥2 mg/L assessed before first administration of an IL-1beta antagonist, and wherein said patient has a reduced hsCRP level of <2.3 mg/L assessed at a predetermined time point after the first administration of said IL-1beta antagonist.

2. A method for reducing risk of progression of OA and/or reducing adverse events associated with OA in a patient, comprising administering an IL-1beta antagonist, wherein said patient has a high sensitivity C-reactive protein (hsCRP) level of ≥2 mg/L assessed before first administration of an IL-1beta antagonist, and wherein said patient will continue to receive IL-1beta antagonists, provided said patient has a reduced hsCRP level of <2.3 mg/L assessed at a predetermined time point after first administration of said IL-1beta antagonist.

3. A method for reducing risk of progression of OA and/or reducing adverse events associated with OA in a patient, comprising administering canakinumab, wherein said patient has a high sensitivity C-reactive protein (hsCRP) level of ≥2 mg/L assessed before first administration of canakinumab, and wherein said patient has a reduced hsCRP level of <2.3 mg/L assessed at about 3 months or more after the first administration of canakinumab.

4. A method for reducing risk of progression of OA and/or reducing adverse events in a patient, comprising administering canakinumab, wherein said patient has a high sensitivity C-reactive protein (hsCRP) level of ≥2 mg/L assessed before first administration of canakinumab, and wherein said patient will continue to receive canakinumab, provided said patient has a reduced hsCRP level of <2.3 mg/L assessed at about 3 months or more after the first administration of canakinumab.

5. The method of any of the preceding claims, wherein said progression of OA include joint replacement.

6. The method of any of the preceding claims, wherein said patient has documented and/or symptomatic OA.

7. The method of any of the preceding claims, comprising administering 150 mg to 300 mg canakinumab.

8. The method according to any of the preceding claims, comprising administering 150 mg canakinumab.

9. The method according to any of the preceding claims, comprising administering 150 mg canakinumab approximately every 3 months.

10. The method according to any of the preceding claims, wherein the reduced level of hsCRP assessed approximately 3 months after first administration of canakinumab or after a predetermined timepoint after first administration of an IL-1beta antagonist is <1.5 mg/L.

11. The method according to any of the preceding claims, wherein the reduced level of hsCRP assessed approximately 3 months after first administration of canakinumab or after a predetermined timepoint after first administration of an IL-1beta antagonist is <1.0 mg/L

12. The method according to any of the preceding claims, wherein the reduced level of hsCRP assessed approximately 3 months after first administration of canakinumab or after a predetermined timepoint after first administration of an IL-1beta antagonist is <2.2, <2.1, <2.0, <1.9, <1.8, <1.7, <1.6, <1.5, <1.4, <1.3, <1.2, <1.1, <1.0, <0.9, <0.8, <0.7, <0.6, or <0.5 mg/L.

13. The method according to any of the preceding claims, wherein the documented OA has been assessed using X-ray and/or MRI.

14. The method according to any of the preceding claims, wherein the symptomatic evidence of OA is pain and/or impaired function.

15. The method according to any of the preceding claims, wherein the patient is not eligible for surgery.

16. The method according to any of the preceding claims, wherein the patient is not responsive to NSAIDs.

17. The method according to any of the preceding claims, wherein the levels of IL-6 after a predetermined timepoint after first administration of an IL-1beta antagonist or 3 months after first administration of canakinumab is below 1.15 mg/L or below 2 mg/L.

18. The method according to any of the preceding claims, wherein said patient previously has suffered a CV event.

19. The method according to any of the preceding claims, wherein said patient previously has suffered a myocardial infarction.

20. A method for reducing risk of progression of OA and/or reducing adverse events associated with OA in a patient, comprising administering an IL-1beta antagonist, wherein said patient has a high sensitivity C-reactive protein (hsCRP) level of ≥2 mg/L assessed before first administration of said IL-1beta antagonist.

21. The method according to claim 20, wherein the IL-1beta antagonist is canakinumab.

22. The method of claim 20 or 21, comprising administering 150 mg to 300 mg canakinumab.

23. The method according to any of the claims 20-22, comprising administering 150 mg to 300 mg canakinumab approximately every 3 months.

24. The method according to any of the claims 20-23, wherein the documented OA has been assessed using X-ray and/or MRI.

25. The method according to any of the claims 20-24, wherein the symptomatic evidence of OA is pain and/or impaired function.

26. The method according to any of the claims 20-25, wherein the patient is not eligible for surgery.

27. The method according to any of the claims 20-26, wherein the patient is not responsive to NSAIDs.

28. The method according to any of the claims 20-27, wherein said patient previously has suffered a CV event.

29. The method according to any of the claims 20-28, wherein said patient previously has suffered a myocardial infarction.

30. The method according to any of the preceding claims, wherein said total joint replacement can be total knee replacement or total hip replacement.

31. The method according to any of the preceding claims, wherein the patient suffers from shoulder OA, hand OA, or spondylarthrosis (degenerative spinal joint disease).

32. The method according to any of the preceding claims, where the total joint replacement can be total shoulder replacement.

33. The method according to any of the claims 1-2 and 7-9, wherein the predetermined time point is 2 weeks up to 6 months.

34. The method according to any of the claims 1-2 and 7-9, wherein the predetermined time point is 4 weeks to 12 weeks.


SEQUENCE LISTING (18 sequences)

SEQ ID NO: 1 - protein, 11 aa, Artificial Sequence
  Light chain CDR1 sequence
RASQSIGSSLH

SEQ ID NO: 2 - protein, 6 aa, Artificial Sequence
  Light chain CDR2 sequence
ASQSFS

SEQ ID NO: 3 - protein, 7 aa, Artificial Sequence
  Light chain CDR3 sequence
HQSSSLP

SEQ ID NO: 4 - protein, 107 aa, Artificial Sequence [sequence omitted]

SEQ ID NO: 5 - protein, 5 aa, Artificial Sequence
  Heavy chain CDR1 sequence
VYGMN

SEQ ID NO: 6 - protein, 17 aa, Artificial Sequence
  Heavy chain CDR2 sequence
IIWYDGDNQYYADSVKG

SEQ ID NO: 7 - protein, 6 aa, Artificial Sequence
  Heavy chain CDR3 sequence
DLRTGP

SEQ ID NO: 8 - protein, 118 aa, Artificial Sequence [sequence omitted]

SEQ ID NO: 9 - protein, 11 aa, Artificial Sequence
  Light chain CDR1 sequence
RASQDISNYLS

SEQ ID NO: 10 - protein, 7 aa, Artificial Sequence
  Light chain CDR2 sequence
YTSKLHS

SEQ ID NO: 11 - protein, 9 aa, Artificial Sequence
  Light chain CDR3 sequence
LQGKMLPWT

SEQ ID NO: 12 - protein, 107 aa, Artificial Sequence [sequence omitted]

SEQ ID NO: 13 - protein, 7 aa, Artificial Sequence
  Heavy chain CDR1 sequence
TSGMGVG

SEQ ID NO: 14 - protein, 15 aa, Artificial Sequence
  Light chain CDR2 sequence
HIWWDGDESYNPSLK

SEQ ID NO: 15 - protein, 10 aa, Artificial Sequence
  Light chain CDR3 sequence
NRYDPPWFVD

SEQ ID NO: 16 - protein, 120 aa, Artificial Sequence [sequence omitted]

SEQ ID NO: 17 - protein, 214 aa, Artificial Sequence
  Light chain sequence
EIVLTQSPDFQSVTPKEKVTITCRASQSIGSSLHWYQQKPDQSPKLLIKYASQSFSGVPSRFSGSGSGTDFTLTINSLEAEDAAAYYCHQSSSLPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 448 aa, Artificial Sequence
  Heavy chain sequence
QVQLVESGGGVVQPGRSLRLSCAASGFTFSVYGMNWVRQAPGKGLEWVAIIWYDGDNQYYADSVKGRFTISRDNSKNTLYLQMNGLRAEDTAVYYCARDLRTGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK